(19) United States
(12) Patent Application Publication
(10) Pub. No.: US 2010/0047284 A1
(43) Pub. Date: Feb. 25, 2010

(54) METHOD FOR REDUCING SPASMS COMPRISING TREATMENT WITH AN AGENT THAT ELEVATES THE LEVEL OF ONE OR MORE CYCLIC NUCLEOTIDES IN THE MUSCLE

(75) Inventors: David Taggart, Oxon (GB); Antony Galione, Oxon (GB)

Correspondence Address: SEED INTELLECTUAL PROPERTY LAW GROUP PLLC, 701 FIFTH AVE, SUITE 5400, SEATTLE, WA, 98104, US

(73) Assignee: ISIS INNOVATION LIMITED, Oxford (GB)

(21) Appl. No.: 12/373,652

(22) PCT Filed: Jul. 12, 2007
(86) PCT No.: PCT/GB2007/002594

§ 371 Date: Oct. 29, 2009

(30) Foreign Application Priority Data
Jul. 13, 2006 (GB) 0613856.4

(51) Int. Cl. A61K 39/02; A61K 38/45

(52) U.S. Cl. 424/236.1; 424/94.5

(57) ABSTRACT

This invention relates to methods for preventing or reducing spasms in smooth muscle, using an agent that elevates the level of one or more nucleotide in the muscle, or an agent which comprises a toxin. The invention also relates to the use of such agents in the manufacture of a medicament for preventing or reducing spasms in smooth muscle.

The present invention relates to methods and medicaments for preventing or reducing spasms in smooth muscle.

During the past decade or so coronary artery by-pass operations have become a routine procedure. A coronary artery by-pass operation is undertaken when there is a significant narrowing or blocking of one or more of the coronary arteries and thus blood flow to the heart muscle is restricted. The operation creates new routes for the blood to flow around a narrowed or blocked coronary artery thereby allowing sufficient blood flow to deliver oxygen and nutrients to the heart muscle.

A coronary artery by-pass operation involves the grafting of a vessel, usually part of another artery or vein, between the aorta and a position beyond the narrowed or blocked coronary artery. The graft allows blood flow to by-pass the narrowed or blocked coronary artery.

Around 500,000 grafts/coronary artery by-pass operations are now being undertaken per annum worldwide. Of these 500,000 grafts around 15% (that is, about 75,000) fail due to the grafted vessel undergoing vasospasm within the first 24-48 hours following surgery. Vasospasm of the grafted vessel causes the vessel to become occluded which then requires further treatment, usually in the form of drug therapy, angioplasty and/or surgery. In some cases vasospasm of the grafted vessel can cause a heart attack, which sometimes can cause death.

An aim of the present invention is to provide a method to prevent or reduce vasospasm in vessels used in grafts in coronary artery by-pass operations.

According to a first aspect, the present invention provides a method for preventing or reducing spasms in smooth muscle comprising treating the smooth muscle with an agent that elevates the level of one or more cyclic nucleotide in the muscle.

The cyclic nucleotide may be cAMP or cGMP. Preferably the agent causes the level of cAMP in the muscle to be increased.

The agent may increase cyclic nucleotide levels by causing an increase in production of cyclic nucleotides, or by preventing or reducing the degradation of cyclic nucleotides.

Preferably cAMP levels are increased for at least 2 hours, at least 12 hours, at least 24 hours, at least 2 days, at least a week or more.

Preferably the agent is a physiological antagonist. Alternatively the agent may be an irreversible agonist of a receptor which causes cyclic nucleotide levels to increase.

The agent may be an enzyme, such as an ADP-ribosyl transferase.

The agent may be a toxin. The toxin may be as described with reference to the second aspect to the invention.

According to a second aspect, the present invention provides a method for preventing or reducing spasms in smooth muscle comprising treating the smooth muscle with an agent which comprises a toxin.

Preferably the toxin acts to stimulate relaxation of the smooth muscle.

Preferably the toxin is an A-B toxin.

Preferably the toxin is an enzyme. The toxin may be an ADP-ribosyl transferase.

The toxin may act by modifying the alpha subunit of one or more G proteins.

The toxin may elevate the level of on or more cyclic nucleotide in the muscle. Accordingly all the preferred features of the first aspect of the invention may also apply to the second aspect of the invention.

Preferably the toxin modifies the cAMP regulatory pathway. Preferably the toxin causes an increase in cAMP levels in treated muscle.

Preferably the toxin is a bacterial toxin. The toxin may be selected from the group comprising the cholera toxin and the pertussis toxin, or a combination thereof.

The toxin may prevent or reduce smooth muscle spasms by stimulating the activation of adenylyl cyclase in the smooth muscle cells which then catalyzes the conversion of ATP to 3′,5′-cyclic AMP (cAMP) and pyrophosphate. The maintained production of cAMP is believed to lead to a prolonged physiological antagonism which results in the inhibition of smooth muscle contraction and prevents or reduces spasms.

Cholera toxin has an intrinsic ADP-ribosyl transferase activity that causes persistent activation of adenylyl cyclase.

The cholera toxin is a hexameric protein composed of a single A subunit (MW 27,234) and five B subunits (MW 11,677 each). The five subunits are arranged in a pentameric ring with apparent 5-fold symmetry. The A subunit is synthesised as a single polypeptide which is proteolytically nicked during secretion from the bacterium to give rise to di-sulphide linked polypeptides A1 (MW 21,826) and A2 (MW 5,407). It is the A1 fragment of the A subunit, released by a disulphide reduction, that acts enzymatically within the target cells as an ADP-ribosyl transferase. The entire cholera oligomer (MW 85,260) is preferred for toxic activity, as the pentamer of B subunits allows the A subunit to enter the smooth muscle cells. Entry of the toxin into the target cell is caused by the interaction of the intact pentamer of B-subunits (known as the choleragenoid) with the ganglioside GM1 membrane receptor. Once inside a cell the A subunit is reduced releasing the A1 subunit which activates adenylyl cyclase.

The cholera toxin may be used as an intact hexameric protein. Alternatively the A and B subunits may be applied separately, or mixed just before application, and may form the intact hexameric protein in use.

In another embodiment just the A subunit and/or the A1 subunit may be used.

The toxin may be the naturally occurring protein or it may be a recombinantly engineered or synthetic protein. The whole toxin may be used, or a part of the toxin protein may be used. If only a part of the toxin protein is used it must be sufficient to cause a reduction or the prevention of smooth muscle spasms.

A recombinant or synthetic toxin may be identical to the natural protein, or a part thereof, or it may include mutations provided that the resulting protein has a similar or improved ability to reduce or prevent smooth muscle spasms.

The following description applies to at least the first and second aspects of the invention.

Spasms in smooth muscle are caused by the contraction of the smooth muscle cells. Typically the contraction is involuntary. The contraction may be abrupt, forceful and/or prolonged. The contraction may last from several minutes to hours.

The smooth muscle may be in a blood vessel, such as an artery or a vein.

Preferably the blood vessel is an artery or vein from the arm, leg or chest. The blood vessel may be part of the radial artery from the arm, or the saphenous vein from the leg, or a mammary artery. Preferably the blood vessel is treated according to the method of the first or second aspect of the invention before use in a coronary artery by-pass graft.

The spasm prevented or reduced by the method of the invention may be a vasospasm.

Preferably the method of the invention is performed extracorporeally (ex vivo), that is the smooth muscle is treated extracorporeally with an effective amount of the agent.

Preferably the smooth muscle is removed from a patient and then treated extracorporeally according to the method of the invention. Preferably, all steps of the method of the first and second aspect of the invention are performed extracorporeally, that is, on smooth muscle removed from an organism. The organism which provides the smooth muscle may be a human or non-human animal.

If the smooth muscle to be treated is a blood vessel preferably the blood vessel is removed from a donor and treated with an effective amount of the agent outside the body, preferably it is then returned to the body. In the case of a blood vessel for use in a coronary artery by-pass graft, the donor of the blood vessel may be the patient who is to receive the graft, although this need not be the case.

Smooth muscle spasms in blood vessels used as grafts in coronary heart by-pass operations are thought to be due to a number of factors. These factors include the inflammatory and stress responses associated with the surgery which may lead to a surge of vasoactive mediators in the post-operative period. Vasoconstrictor agents such as endothelin, thromboxane A2, prostaglandins, angiotensin II, noradrenaline and adrenaline are all increased soon after surgery and remain elevated for 24-48 hours after surgery. During this time grafts that have been deinnervated during the harvesting process are particularly prone to vasospasm. Patients undergoing cardiac surgery almost always have a degree of myocardial stunning immediately after surgery. This leads to lower cardiac output states and poorer blood flow down the by-pass grafts. Poor blood flow down the grafts may lead to less intrinsic nitric oxide production and thus makes the grafts more prone to spasm. Similarly patients with poor left ventricular function may require exogenous inotropes and vasoconstrictors (e.g. noradrenaline and adrenaline) in order to increase peripheral perfusion pressure. Patients on adrenaline and noradrenaline infusions in the recovery period are even more likely to suffer the damaging effects of graft spasm. Graft spasm in the longer term is likely to increase the chances of early graft occlusion, since the lumen of the graft is narrowed, the impedance to blood flow is increased and blood flow is reduced. Slow moving or stagnant blood is more likely to cause thrombosis in the graft and therefore graft occlusion.

In an alternative embodiment, the smooth muscle may be treated in vivo to cause local paralysis of the muscle. Local treatment may be achieved by topically applying the agent, or by injecting the agent, for example intramuscularly, into the smooth muscle to cause local paralysis.

Preferably the effect of the agent on the smooth muscle is prolonged, and lasts at least 2 hours, at least 12 hours, at least 24 hours, at least 2 days, at least one week or more after administration. Preferably the effect of the agent remains even after exposure to the agent has ceased, the effect may persist for at least 2 hours, at least 12 hours, at least 24 hours, at least 2 days, at least one week or more, after exposure to the agent has ceased.

The amount of agent used is an amount that is sufficient to reduce or prevent spasms in the smooth muscle treated.

Preferably the smooth muscle is treated with the agent at a concentration of from about 1 μg/ml to about 1000 μg/ml, more preferably the concentration of the agent is from about 10 μg/ml to about 500 μg/ml, preferably from about 10 μg/ml to about 100 μg/ml.

The agent may be dissolved or suspended in a liquid solution. This solution can then be applied directly to the smooth muscle, or to a sample containing the smooth muscle, for example, the smooth muscle may be part of an artery or vein. Preferably the smooth muscle to be treated is placed in bath containing the agent in a liquid solution. Preferably the smooth muscle is submerged in a bath of solution containing the agent.

Alternatively, the agent may be provided in a gel or cream which can be applied to the smooth muscle, or to a sample containing smooth muscle.

The agent may be applied topically to the smooth muscle to be treated.

The solution, cream or gel, or any other composition containing the agent may also include pharmaceutically acceptable excipients, these may be selected from the group comprising binders, disintergrants, diluents, lubricants, glidants, compression aids, colours, preservatives, suspending or dispersing agents and other agents well known in the art.

The smooth muscle may be treated with one concentration of the agent, or the concentration may be varied over the course of the treatment in order to achieve the desired result.

Preferably the smooth muscle is treated with the agent for from about 1 minute to about 180 minutes. More preferably from about 10 minutes to about 120 minutes, preferably from about 30 minutes to about 90 minutes.

Preferably treatment with the agent reduces contraction in the smooth muscle by from about 10% to about 95%, more preferably contraction is reduced by from about 10% to about 90%, preferably from about 10% to about 75%, preferably from about 10% to about 60%, preferably from about 10% to about 50%, preferably from about 10% to about 40%. The percentage reduction in contraction can be determined by comparing the contraction of the smooth muscle in response to a constriction agent with and without treatment with the agent. If the smooth muscle is a blood vessel a vasoconstrictor, such as angiotensin II, adrenaline or prostaglandin F2α, may be used to determine the percent reduction in contraction.

Preferably, when the agent is used to treat a blood vessel for use in a coronary artery by-pass graft, the risk of spasm in the graft is reduced by from about 10% to about 90%, more preferably from about 10% to about 80%, preferably from about 10% to about 70%, preferably from about 20% to about 70%, preferably from about 30% to about 70%.

The method of the invention may be used to treat an isolated section of a blood vessel for use as a graft in a coronary artery by-pass. Wherein isolated means that the section of blood vessel is removed from the human or non-human animal donor, and then treated outside the body. Preferably the blood vessel to be used in the graft is isolated from the patient who is to receive the graft. Preferably the blood vessel is treated with the agent prior to being used in the graft. Preferably, by treating the blood vessel with the agent the chance of vasospasm is reduced.

Preferably after the smooth muscle is treated with the agent it is washed one or more times to remove excess agent. Preferably, after washing, at least 25% of the agent is removed, preferably at least 30% is removed, preferably at least 50% is removed, preferably at least 60% is removed, preferably at least 70% is removed, preferably at least 80% is removed, preferably at least 90% is removed, preferably at least about 99.9% of the agent is removed. Preferably after the final wash very little or no agent is detectable in the wash solution.

Preferably the smooth muscle is washed in a physiological buffer, such as sterile Ringers solution, after treatment with the agent.

According to a third aspect, the invention provides a method for reducing or preventing spasms in a blood vessel comprising treating the blood vessel with an agent that elevates the level of one or more cyclic nucleotides in the muscle.

According to a fourth aspect, the invention provides a method for reducing or preventing spasms in a blood vessel comprising treating the blood vessel with an agent which comprises a toxin.

All preferred/optional features described with reference to the first and/or second aspect of the invention can be applied to the third and fourth aspects of the invention.

According to a fifth aspect, the invention provides the use of an agent that elevates the level of one or more cyclic nucleotides in smooth muscle in the preparation of a medicament or pharmaceutical composition for reducing or preventing spasms in smooth muscle.

According to a sixth aspect, the invention provides the use of an agent which comprises a toxin in the preparation of a medicament or pharmaceutical composition for reducing or preventing spasms in smooth muscle.

Preferably the medicament or pharmaceutical composition is for application to the smooth muscle ex vivo or extracorporeally. Wherein ex vivo and extracorporeal are used to mean that the smooth muscle is treated outside the body.

Alternatively the medicament or pharmaceutical composition may be used in vivo to cause local paralysis of smooth muscle.

The medicament or pharmaceutical composition may be in a form suitable for topical application to the smooth muscle.

Preferably the medicament or pharmaceutical composition is in the form of a liquid, which may be applied to the smooth muscle by placing the smooth muscle in a bath containing the medicament or composition. The medicament or pharmaceutical composition may be in the form of a liquid, powder or solid which is dissolved or suspended before use.

The medicament or pharmaceutical composition preferably comprises the agent at a therapeutically effective concentration; alternatively the medicament or composition may have to be diluted to have a therapeutically effective concentration of the agent.

Preferably the medicament or pharmaceutical composition also comprises one or more pharmaceutically acceptable excipients.

Preferably if the agent is a toxin it is a bacterial toxin, such as a cholera toxin, a pertussis toxin or a combination thereof.

Preferably the medicament or pharmaceutical composition is for use in reducing or preventing vasospasms in blood vessels.

According a seventh aspect the invention provides a kit for treating smooth muscle to reduce or prevent muscle spasms comprising an agent which comprises a toxin, and/or an agent that elevates the level of one or more cyclic nucleotides in the muscle, and instructions for use.

Preferably the kit is for use on smooth muscle ex vivo.

Preferably, the toxin is a bacterial toxin, such as a cholera toxin, a pertussis toxin or a combination thereof.

Preferably the agent is provided as a liquid solution. The agent may be ready to use or it may have to be diluted or mixed with other reagents before use.

Alternatively, the agent may be provided as a powder or as a tablet/capsule which has to be dissolved or suspended before use. If provided as a powder, the agent may be lyophilised.

In a further embodiment the agent is provided in a cream or gel which can be topically applied to the smooth muscle.

Preferably all compositions provided in the kit are sterile.

Preferably the kit also includes a receptacle in which to place the smooth muscle. Preferably the receptacle is sterile. The receptacle may be provided in the kit with the solution containing the agent already in it.

The receptacle may include one or more wells, with different wells being intended for use in treating more than one sample, and/or for washing samples after they have been treated with the agent.

The kit may also comprise a buffer solution with instructions to use this to wash the smooth muscle after treatment with the agent. Preferably the buffer is sterile. The buffer used may be any suitable physiological buffer. Such buffers are well known to the man skilled in the art.

Preferably the kit is for use with a piece of artery or vein. Preferably the treated artery or vein is for use in a coronary by-pass operation.

The kit may be intended for treating a piece of a blood vessel ex vivo to reduce or prevent vasospasms.

The instructions may include directions to use the agent in the kit according to the method of the first or second aspect of the invention.

According to an eighth aspect, the present invention provides a method of performing a coronary artery by-pass graft comprising:
i) excising a piece of artery or vein from a patient;
ii) treating the excised artery or vein according to the method of the first or second aspect of the invention, to reduce or prevent spasm in the artery or vein; and
iii) grafting the treated artery or vein to the patient to by-pass a blocked or occluded coronary artery.

The skilled man will appreciate that any of the preferred/optional features discussed above can be applied to many of the aspects of the invention. In particular, all the preferred/optional features relating to the nature of the agent described with reference to the first and/or second aspects of the invention can be applied to the fifth, sixth, seventh and eighth aspects of the invention.

Embodiments of the present invention will now be described, by way of example only, with reference to the following figures.

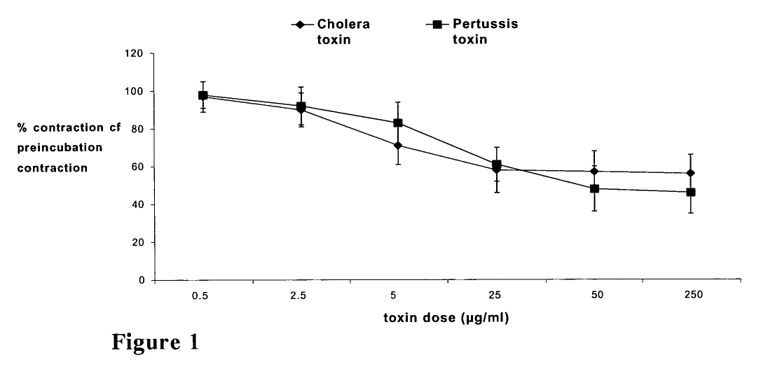

FIG. 1—illustrates adrenaline (5×10⁻⁵ M) mediated contraction of radial artery rings when incubated for 30 minutes with various doses of cholera (n=4) and pertussis (n=4) toxins;

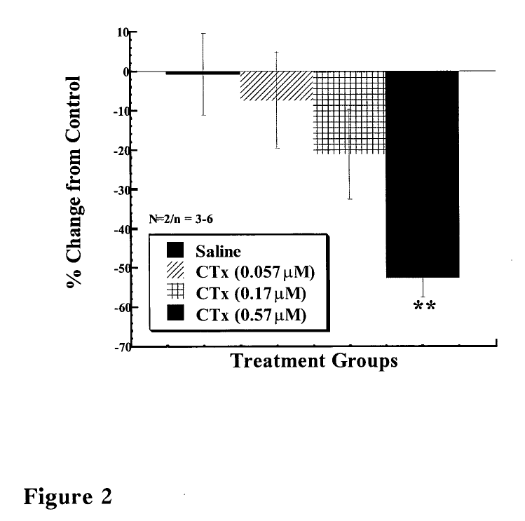

FIG. 2—illustrates the effect of various concentrations of cholera toxin on angiotensin II evoked contraction of mammary artery samples;

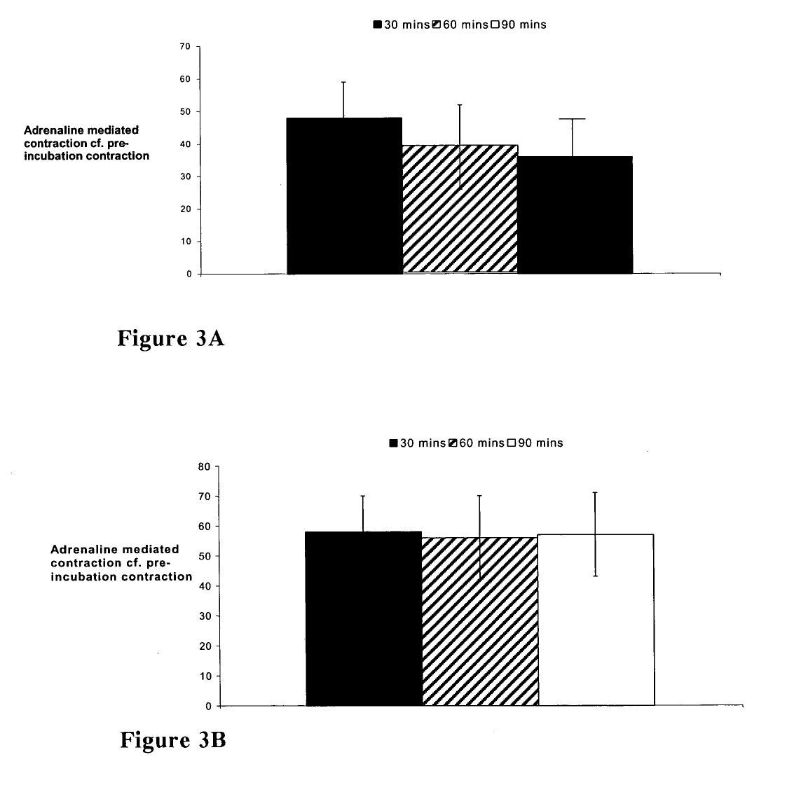

FIG. 3A—illustrates the effect of incubation time on inhibition of adrenaline (10⁻⁶ M) mediated contraction of radial artery rings when incubated with 50 μg/ml pertussis toxin (n=6);

FIG. 3B—illustrates the effect of incubation time on inhibition of adrenaline (10⁻⁶ M) mediated contraction of radial artery rings when incubated with 25 μg/ml cholera toxin (n=6);

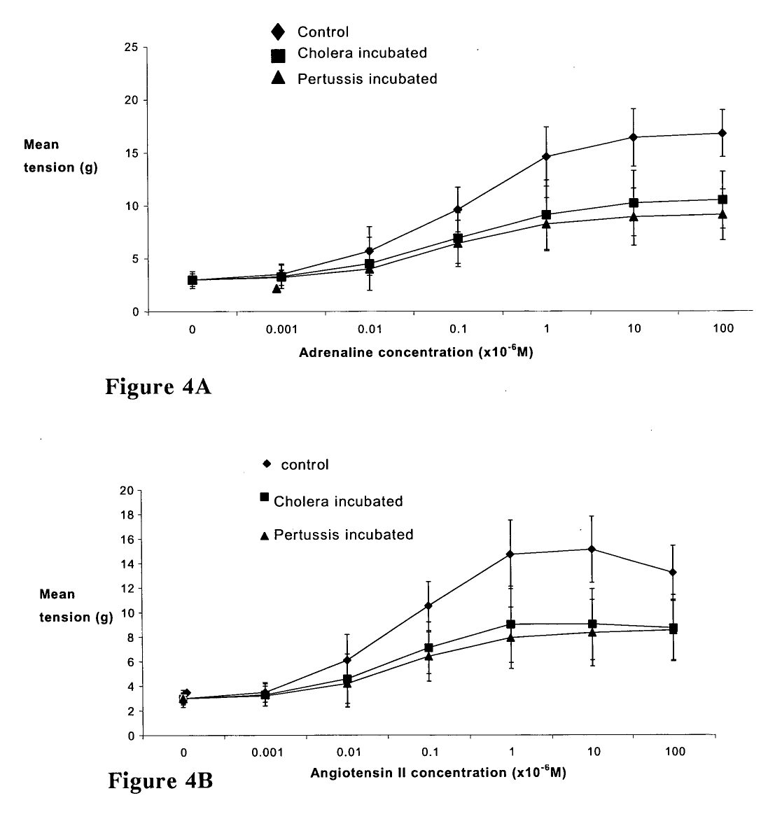

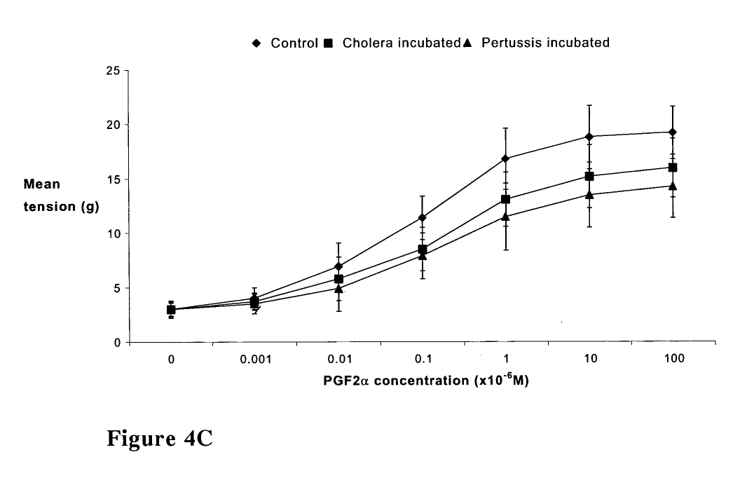

FIG. 4A—illustrates a dose response curve for adrenaline mediated vasoconstriction in radial artery rings incubated with a control, cholera toxin or pertussis toxin (n=12);

FIG. 4B—illustrates a dose response curve for angiotensin II mediated vasoconstriction in radial artery rings incubated with a control, cholera toxin or pertussis toxin (n=10);

FIG. 4C—illustrates a dose response curve for PGF2α mediated vasoconstriction in radial artery rings incubated with a control, cholera toxin or pertussis toxin (n=10);

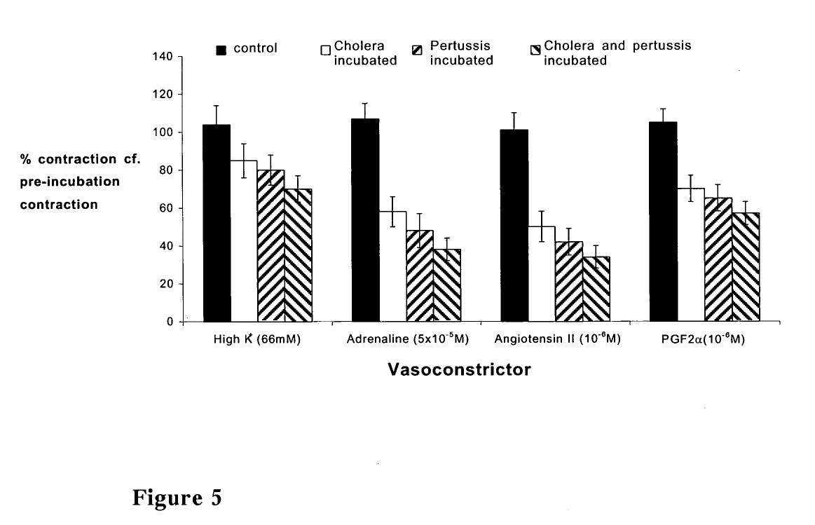

FIG. 5—illustrates the effect of incubating radial artery rings with a control, cholera toxin, pertussis toxin or a combination of cholera and pertussis toxin on subsequent agonist induced vasoconstriction using high K⁺, adrenaline, angiotensin II and PGF2α (n=8);

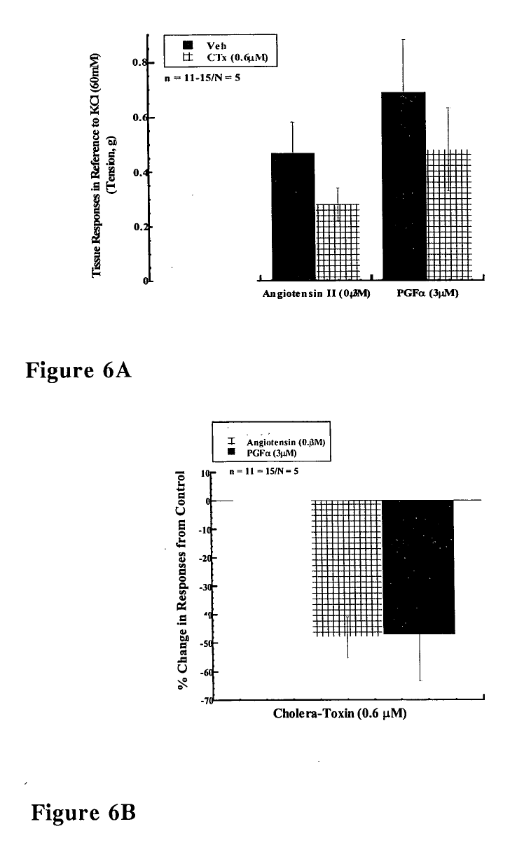

FIG. 6A—illustrates the effect of cholera toxin on angiotensin II and prostaglandin F2 alpha(PGF2α)-induced contractions in mammary artery samples. FIG. 6B shows the percentage change in the response between the samples depicted in FIG. 6A compared to a control with no agonist;

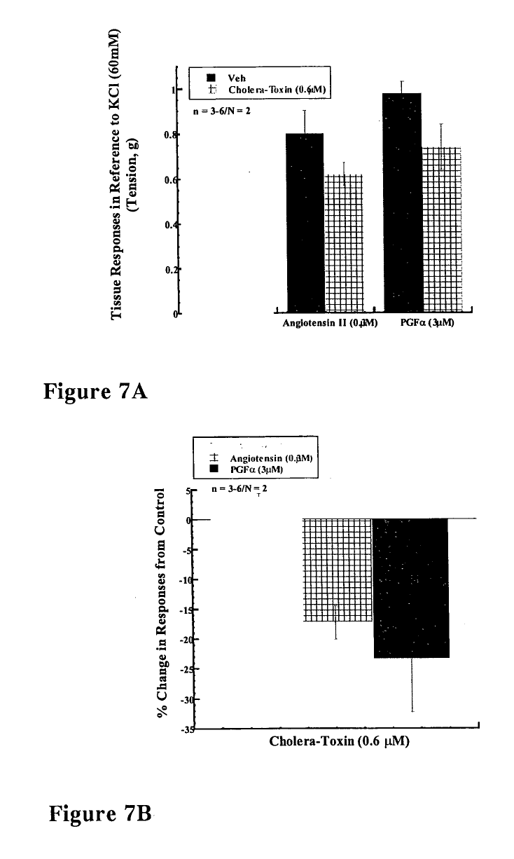

FIG. 7A—illustrates the effect of cholera toxin on angiotensin II and prostaglandin F2 alpha(PGF2α)-induced contractions in radial artery samples. FIG. 7B shows the percentage change in the response between the samples depicted in FIG. 6A compared to a control with no agonist;

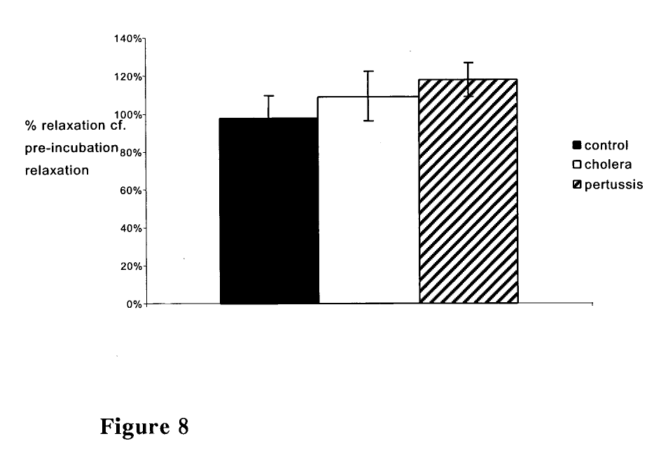

FIG. 8—illustrates the degree of relaxation induced by carbachol (10⁻⁴ M) after maximal contraction to PGF2α;

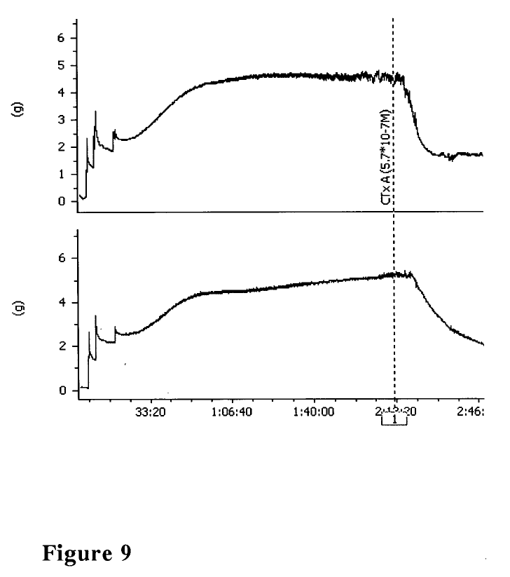

FIG. 9—illustrates two examples of stretch induced contraction in saphenous vein samples, and demonstrates the ability of the cholera holotoxin (CTx) to relax the muscle after a delay consistent with intracellular toxin entry and enzymic-mediated stimulation of activation of the cAMP signalling pathway;

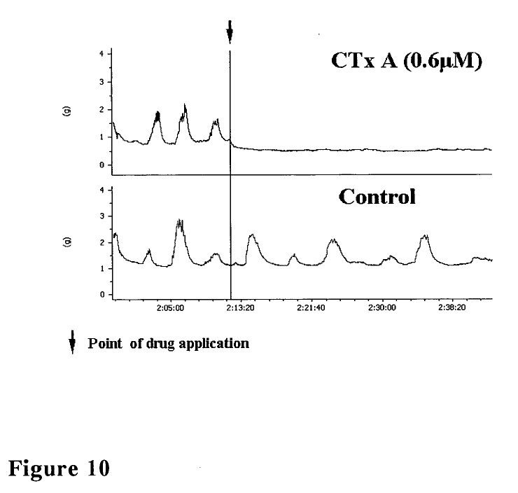

FIG. 10—illustrates initially, for the first third of the graph, the spontaneous contractile activity of mammary artery samples, and demonstrates that this activity is blocked by addition of the cholera toxin;

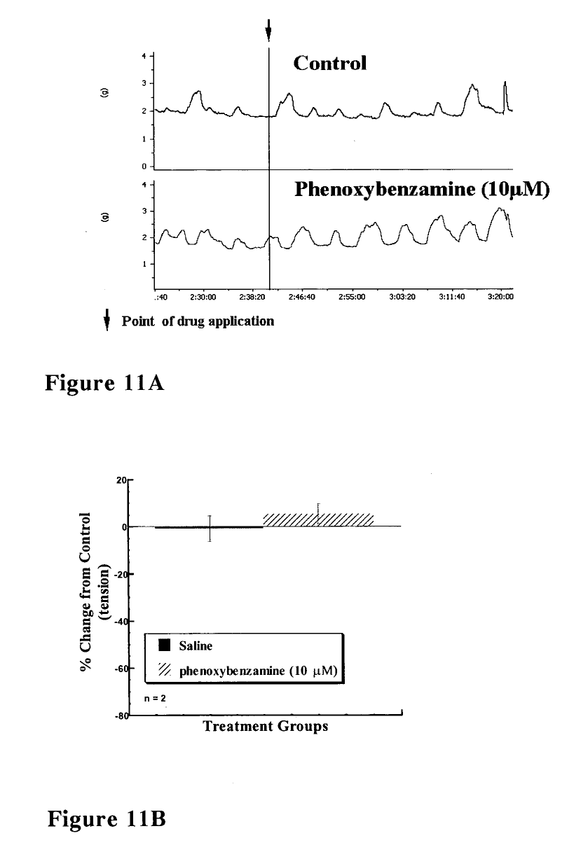

FIG. 11—demonstrates the lack of effect of the irreversible alpha1-adrenoceptor antagonist phenoxybenzamine on the spontaneous contractility of radial artery tissue. FIG. 11B demonstrates that phenoxybenzamine has no effect on angiotensin II-induced contraction in radial artery tissue.

### MATERIALS AND METHODS

### Drugs and Solutions

PSS was freshly prepared for each experiment and was composed of: Na⁺ 137 mmol/L, K⁺ 5.9 mmol/L, Mg²⁺ 1.2 mmol/L, Ca²⁺ 2.5 mmol/L, Cl⁻ 134 mmol/L, H₂PO₄⁻ 1.2 mmol/L, HCO³⁻ 15.5 mmol/L and glucose 11.5 mmol/L, pH 7.4. The following stock solutions of drugs were prepared in PSS and stored in frozen 0.8 ml aliquots: adrenaline (5×10⁻⁴ M), angiotensin II (10⁻⁴ M), prostaglandin F2α/PGF2α (10⁻⁴ M), carbachol (10⁻² M), cholera and pertussis toxins distributed by Quadratech Ltd, Surrey, UK sourced from List Biological Laboratories, Inc, California, USA.

### Specimen Collection and Preparation

Human radial artery (RA) segments were collected from sixty patients undergoing an elective coronary artery by-pass graft (CABG). Radial arteries were harvested using a longitudinal forearm incision, and careful dissection of the artery and its pedicle with scissors, clips and diathermy. A 1-2 cm segment of the most distal portion of the RA was immediately placed in chilled (4° C.) physiological saline solution (PSS) and transferred to the laboratory on ice where fat and periarteriolar connective tissue was removed. In addition to radial artery segments, segments from excised mammary and saphenous veins were also used.

Segments of the isolated vessel wall were cut helically, the endothelium was removed and smooth muscle strips (4×1×1 mm) weighing 2-4 mg were dissected. 5/0 silk ligatures were used to mount the smooth muscle strips vertically between 2 platinum ring electrodes in 0.2 ml superfusion organ bath chambers. A peristaltic pump (Watson-Marlow) and silicone tubing were used to continuously superfuse the muscle strips with Krebs' solution at 1 ml/min, bubbled with 97% O₂ and 3% CO₂, and warmed to 37° C. by a heated water bath (Grant Instruments, Cambridge). Strip tension was measured isometrically using a transducer (Pioden dynamometer UF1 force-displacement transducer; Pioden Controls Ltd., Canterbury, UK), attached to moveable units so that the tension of each strip could be adjusted. At the beginning of each experiment, the strips were tensioned to 1 g, and left to equilibrate for 90 minutes in aerated PSS (95% O₂, 5% CO₂) at 37° C. The tension was then left the same and various test solutions were added. The effect of the test solutions on the contraction of the strips was monitored by monitoring any change in tension. Typically the test solutions used were prostaglandin F2α (10⁻⁶ M), adrenaline (5×10⁻⁵M) or angiotensin II (10⁻⁶ M) (the concentrations of vasocontrictors used were pre-determined from preliminary experiments).

Results were recorded on an Apple Macintosh computer (PowerBook 1400 c) using Chart v3.6 software and PowerLab 800 hardware (AD instruments Pty, New South Wales, Australia). Results are expressed as mean contractions±SEM relative to pre-incubation contractions for the same vasoconstrictor agent. ‘n’ indicates the number of rings. Statistical analysis was performed using ANOVA and p<0.05 was regarded as significant.

An optimal concentration of about 0.6 μM (about 51 μg/ml) cholera toxin was found to be the most effective at reducing contractile activity. Preincubation of CTx of between 45-60 min before washout allowed abolition of spontaneous or inhibition of agonist-induced contraction, for at least 24 hours post-incubation, in all types of vascular smooth muscle studied.

### EXAMPLES

### Determining Effective Concentrations of Cholera and Pertussis Toxins

To determine an effective concentration of cholera and pertussis toxins to use to inhibit smooth muscle spasms, radial artery rings were incubated with different concentrations of cholera and pertussis toxins and the degree of inhibition to adrenaline (5×10⁻⁵ M) was determined (FIG. 1). Cholera toxin was shown to be most effective at a concentration of 25 μg/ml and pertussis toxin at a concentration of 50 μg/ml.

Similar experiments using mammary artery muscle strips demonstrated cholera toxin to be most effective at a concentration of 0.57 μM technique. This is supported by FIG. 2 which shows the results of incubating mammary artery samples with different concentrations of cholera toxin and observing the degree of inhibition of angiotensin II induced contraction.

### Determining Effective Incubation Times for Cholera and Pertussis Toxins

In order to determine the duration of incubation necessary to inhibit subsequent vasoconstriction induced by adrenaline 10⁻⁶ M, radial artery rings were incubated for 30, 60 or 90 minutes with either cholera or pertussis toxins. The results in FIG. 3A (for the pertussis toxin) and 3B (for the cholera toxin) show that cholera is effective after 30 mins and pertussis is effective at 60 mins incubation time.

### Effect of Cholera and Pertussis Toxin Incubation on Dose Response Curves to Adrenaline, Angiotensin II and PGF2α

FIGS. 4A, 4B and 4C show the dose response curves of radial artery rings incubated in either PSS (control), or cholera toxin 25 μg/ml for 30 minutes, or pertussis toxin 50 μg/ml for 60 minutes and adrenaline, angiotensin II or PGF2α respectively. The results show that both the cholera and pertussis toxins inhibited the contraction of the radial artery samples when subjected to various vasoconstrictors.

Effect of Pre-Incubating Radial Artery Rings with Cholera Toxin, Pertussis Toxin and a Combination of Both, on Subsequent Vasoconstrictor Responses

The effect of pre-incubating radial artery rings with either cholera toxin, pertussis toxin or a combination of both on subsequent vasoconstrictor responses was compared (FIG. 5). Both cholera and pertussis toxins inhibit the maximal contraction force produced and the sensitivity of radial artery rings to the vasoconstrictors tested. Cholera inhibits contraction by 15-50% depending on the vasoconstrictor. Pertussis inhibits contraction by between 20-60%. Cholera and pertussis in combination is more effective than cholera or pertussis alone.

The observation that cholera toxin inhibits vasoconstrictor induced contraction in further supported by the results in FIGS. 6A and 6B and FIGS. 7A and 7B.

FIGS. 6A and 6B shows the effects of pre-incubating a mammary artery isolated ring in cholera toxin on the response of the artery to subsequent exposure to angiotensin II or PGFα. Test tissue samples were incubated in 0.6 μM cholera toxin for 1 hour and control tissue samples were incubated for 1 hour in buffer, all samples were then washed and a solution of 0.3 μM angiotensin II or 3 μM PGFα was added to each sample. The samples were allowed to equilibrate in the solutions before commencement of the contraction test. The results in FIGS. 6A and 6B clearly show that cholera toxin dramatically reduces vasoconstrictor induced contraction compared to the control (designated as “veh”) which has not been treated with cholera toxin. An inhibition of between 45 to 50% is observed for both vasoconstrictors.

The results in FIGS. 7A and 7B were obtained as described with reference to FIGS. 6A and 6B except that radial artery samples were used rather than mammary artery samples. Again, the cholera toxin is shown to inhibit angiotensin II and PGFα induced contractions. Inhibition of between about 17 and about 22% was observed.

### Assessment of Endothelial Function

The degree of relaxation induced by the acetylcholine analogue carbachol (CCh) (10⁻⁴ M) after maximal contraction to prostaglandin F2α was recorded and used as a measure of endothelial function, both before and after incubation with cholera and pertussis toxins (FIG. 8). There was no impairment of endothelial mediated relaxation after incubation with either cholera or pertussis toxins.

### Cholera Toxin Abolishes Stretch-Evoked Smooth Muscle Spasms

Cholera toxin was also shown to abolish stretch-evoked contractions. Normal smooth muscle tends to respond to being stretched by contracting. The results in FIG. 9 illustrate that the stretch evoked contraction can be abolished by the addition of cholera (CTx) at 0.6 μM. A sample of saphenous vein was stretched and induced to contract, a 0.6 μM solution of cholera toxin was then added, at point “1” in FIG. 9, and the contraction was abolished and the muscle relaxed. FIG. 9 shows the results for two separate experiments.

### Cholera Toxin Abolishes Spontaneous Smooth Muscle Spasms

The ability of cholera toxin to abolish spontaneous contraction in mammary artery samples was demonstrated. FIG. 10 illustrates the occurrence of spontaneous contractions in mammary artery tissue samples, at the time point indicated by the arrow cholera toxin at a concentration of 0.6 μM was added to one sample and, as can be seen in the graph, spontaneous contractions ceased.

### Failure of Phenoxybenzamine to Abolish Angiotensin Induced Smooth Muscle Contraction

Phenoxybenzamine is currently used in some coronary artery by-pass operations to try and reduce spontaneous contraction of the smooth muscle in the grafted blood vessel. Phenoxybenzamine is understood to work by blocking alpha1-adrenoreceptors on the surface of smooth muscles, thereby inhibiting or preventing adrenaline induced contractions. The data presented in FIGS. 11A and 11B illustrates that phenoxybenzamine does not inhibit smooth muscle contraction in response to angiotensin II. Thus demonstrating the benefit of using a physiological antagonist such as the cholera or pertussis toxin which works from within the cell and not via a single cell surface receptor. The data presented in FIG. 11A illustrates the effect, or lack of effect, of phenoxybenzamine on angiotensin II induced contraction of radial artery tissue. As can be seen the addition of phenoxybenzamine has no effect on spontaneous contraction of the samples. FIG. 11B shows the results of suspending a sample of radial artery tissue in 10 μM phenoxybenzamine for 45 minutes, then washing the tissue and suspending it in a solution of 0.3 μM angiotensin II. As can be seen from FIG. 11B pre-incubation in phenoxybenzamine prior to addition of angiotensin II has no inhibitory effect on contraction of the tissue.

1. A method for preventing or reducing spasms in smooth muscle comprising treating the smooth muscle with an agent that elevates the level of one or more cyclic nucleotides in the muscle.

2. (canceled)

3. The method of claim 1 wherein the cyclic nucleotide is cAMP.

4. (canceled)

5. The method of claim 1 wherein the agent is a toxin.

6.-11. (canceled)

12. The method of claim 1 wherein the toxin is a bacterial toxin.

13. The method of claim 12 wherein the toxin comprises (a) cholera toxin, (b) pertussis toxin, or (c) a combination of cholera toxin and pertussis toxin.

14.-15. (canceled)

16. The method of claim 1 wherein the agent is an enzyme.

17. The method of claim 16 wherein the enzyme is an ADP-ribosyl transferase.

18.-19. (canceled)

20. The method of claim 1 wherein the smooth muscle is a blood vessel or a part thereof.

21.-29. (canceled)

30. The method of claim 1 wherein the smooth muscle is an isolated section of a blood vessel, and wherein the isolated section is a graft in a coronary artery by-pass.

31.-32. (canceled)

33. A kit for treating smooth muscle to reduce or prevent muscle spasms wherein said kit comprises (a) a toxin, and an agent that elevates the level of one or more cyclic nucleotides in the muscle, and (b) instructions for use.

34.-43. (canceled)

44. A method for preventing or reducing spasms in smooth muscle comprising treating the smooth muscle with an agent wherein the agent comprises a toxin.

45. The method of claim 44 wherein the toxin is an A-B toxin.

46. The method of claim 44 wherein the toxin elevates the level of one or more cyclic nucleotides in the muscle.

47. The method of claim 44 wherein the toxin is a bacterial toxin.

48. The method of claim 47 wherein the toxin comprises (a) cholera toxin, (b) pertussis toxin, or (c) a combination of cholera toxin and pertussis toxin.

49. The method of claim 44 wherein the agent comprises an enzyme.

50. The method of claim 49 wherein the enzyme is an ADP-ribosyl transferase.

51. The method of claim 44 wherein the smooth muscle is a blood vessel or a part thereof.

52. The method of claim 44 wherein the smooth muscle is an isolated section of a blood vessel, and wherein the isolated section is a graft in a coronary artery by-pass.
